(19) United States
(12) Patent Application Publication
(10) Pub. No.: US 2023/0346347 A1
(43) Pub. Date: Nov. 2, 2023

(54) FLUID-FILLED CATHETER AND RESERVOIR

(71) Applicant: ACIST Medical Systems, Inc., Eden Prairie, MN (US)

(72) Inventor: Yun Zhou, Eden Prairie, MN (US)

(21) Appl. No.: 17/731,465

(22) Filed: Apr. 28, 2022

(51) Int. Cl. A61B 8/00; A61B 5/00; A61N 7/00; A61B 8/08

(52) U.S. Cl. CPC A61B 8/4422; A61B 8/4281; A61B 5/0084; A61N 7/00; A61B 8/481; A61N 2007/0039; A61M 5/315

(57) ABSTRACT

A method of using a catheter includes removing a packaging enclosing the catheter and a fluid reservoir. The catheter removed from the packaging includes a catheter housing defining an interior lumen, an ultrasound transducer disposed within the interior lumen, and a first fluid medium within the interior lumen. The fluid reservoir removed from the packaging includes a second fluid medium. After removing the packaging, a fluid outlet is created at the interior lumen and the catheter is connected to an imaging engine and delivered to a region of interest. Ultrasound energy is emitted from the ultrasound transducer into the region of interest. At least a portion of the second fluid medium is delivered from the fluid reservoir to the interior lumen and at least a portion of the first fluid medium is thereby expelled from the interior lumen through the fluid outlet.

### TECHNICAL FIELD

This disclosure relates generally to catheters and associated packaging assemblies used during certain medical imaging procedures, and methods of using such catheters and associated fluid reservoirs.

### BACKGROUND

Medical imaging techniques generally can be used to collect data and generate in-vivo visualization of anatomical areas of interest. One such example is intravascular imaging, where images of vascular structures and lumens may be generated. For instance, intravascular imaging may be used to produce one or more images of the coronary artery lumen, of the coronary artery wall morphology, and of devices, such as stents, at or near the coronary artery wall. Images generated using medical imaging techniques can be useful for diagnostic purposes, such as identifying diagnostically significant characteristics of a vessel, and/or in guiding an interventional procedure.

To collect image data, intravascular imaging procedures generally use an imaging catheter that is inserted within a vascular structure. Before image data is collected, steps are sometimes taken to prepare the catheter for use. For example, a clinician may flush an interior portion of the catheter with a flushing solution prior to using the catheter and/or after an initial use of the catheter to facilitate and/or enhance the imaging procedure or the quality of the images obtained during the procedure. This can, however, increase user burden and time associated with using the catheter.

### SUMMARY

This disclosure in general provides embodiments relating to a fluid-filled imaging catheter that can begin collecting imaging data (e.g., immediately) upon connecting the imaging catheter to an imaging engine.

As one example, a catheter housing can define an interior lumen that is filled with a first fluid medium, such as an acoustic coupling fluid medium. This first fluid medium may surround an ultrasound transducer positioned within a portion of the interior lumen. The described catheter housing can be packaged for a prolonged period of time (e.g., months) without detrimentally affecting properties of the ultrasound transducer. In addition, when the catheter housing is removed from the packaging at a later time, said fluid medium can serve to transmit ultrasound energy between the transducer and the surrounding vessel in a manner that can facilitate collection of high quality image data. Such embodiments can provide a variety of useful advantages, including a reduction in user burden, setup time, and/or time needed to collect image data. Such advantages may be particularly useful in intravascular imaging applications, where it can be desirable to reduce both the imaging procedure time as well as the number of accessories and interconnections required during setup in a sterile environment.

Other benefits may include reduced user error and user training. For a single element rotational catheter, even with a well-trained user, there is still a chance of failing to perform the catheter flush, leaving bubbles in the vicinity of the transducer and creating poor image quality as a result. Imaging catheters, such as those used for intravascular ultrasound (“IVUS”) and/or optical coherence tomography (“OCT”), may be pre-filled with fluid, which can reduce the need to train a user how to properly pre-flush catheters and/or to identify the image artifacts from residual bubbles.

In further embodiments, the present disclosure more specifically describes a fluid reservoir accompanying the fluid-filled imaging catheter, for instance, to facilitate delivery of a fluid medium from the fluid reservoir to the imaging catheter. As one example, the accompanying fluid reservoir can facilitate flushing of the interior lumen of the catheter. In some cases, the fluid-filled catheter housing can accumulate bubbles over time, such as prior to use while packaged, for instance as a result of air permeating into the interior lumen through an imaging window of the catheter housing, and/or during use, for instance as a result of movement of the ultrasound transducer within the interior lumen. The accompanying fluid reservoir can thus help to flush any such bubbles away from the ultrasound transducer by providing the ability to deliver the fluid medium from the fluid reservoir to the interior lumen of the catheter.

In some embodiments of this disclosure, a system is described comprising an imaging catheter having a catheter housing defining an interior lumen that is filled with a first fluid medium. The imaging catheter may be any of a variety of imaging catheter types, such as those used for intravascular ultrasound (“IVUS”) imaging and/or optical coherence tomography (“OCT”) imaging, or other similar types of imaging technologies in which an energy source emits energy that is useful in generating image data. In the case of IVUS imaging, the energy source may be an ultrasound transducer, for example. In the case of OCT imaging, the energy source may be a light source (e.g., superluminescent diodes, ultrafast lasers, supercontinuum sources, swept sources, optical fibers, etc.) that transmits light energy. The system may further comprise a fluid reservoir accompanying the fluid-filled imaging catheter to facilitate delivery of a second fluid medium from the fluid reservoir to the imaging catheter. In some embodiments of a system, the imaging catheter and fluid reservoir may be fluidly coupled such that they are in fluid communication. For example, the fluid reservoir may be used to facilitate flushing of the interior lumen of the imaging catheter. In some embodiments of the system, such flushing may help to remove air/gas bubbles away from the imaging energy source (e.g., the ultrasound transducer, light source, etc.) in preparation for performing an imaging procedure.

As another example, the accompanying fluid reservoir can facilitate delivering a therapy in conjunction with use of the fluid-filled imaging catheter. In some cases, the fluid reservoir can include a fluid medium for use in a therapy, for instance the fluid medium may include microbubbles that include a medication. The fluid-filled imaging catheter can be used to generate image data of a region of interest. The image data of the region of interest can then be used to guide the therapy, such as to determine whether the fluid medium (e.g., microbubbles including a medication) from the fluid reservoir should be applied. Upon determining that the therapy should be applied, the fluid medium from the fluid reservoir can be delivered to the region of interest. In other cases, the fluid reservoir can include a fluid medium that serves as a cooling fluid for use with an ablation procedure. The fluid-filled catheter could, for example, be used to deliver ablation energy (e.g., via an ultrasound transducer) to a region of interest, and the fluid medium from the fluid reservoir could be delivered to cool one or more areas associated with the ablation procedure (e.g., the interior lumen, the region of interest, etc.).

One exemplary embodiment includes a method of using an imaging catheter. This method embodiment includes the step of removing a packaging enclosing the catheter and a fluid reservoir. The catheter removed from the packaging includes a catheter housing defining an interior lumen, an ultrasound transducer disposed within the interior lumen, and a first fluid medium within the interior lumen. The fluid reservoir removed from the packaging includes a second fluid medium and is in fluid communication with the interior lumen of the catheter. This method embodiment may further include the steps of, after removing the packaging, creating a fluid outlet at the interior lumen, connecting the catheter to an imaging engine, and delivering the catheter to a region of interest within a patient. The method embodiment may also include the step of emitting ultrasound energy from the ultrasound transducer into the region of interest. Additionally, the method embodiment includes the step of delivering at least a portion of the second fluid medium from the fluid reservoir to the interior lumen and thereby displacing (e.g., removing) at least a portion of the first fluid medium from the interior lumen through the fluid outlet.

In a further embodiment of the method, creating the fluid outlet at the interior lumen includes opening an aperture defined at the catheter housing. As one example, opening the aperture defined at the catheter housing includes removing a plug from the aperture. In an alternate embodiment, creating the fluid outlet at the interior lumen includes creating or forming an aperture or opening in the catheter housing.

In a further embodiment of the method, delivering at least a portion of the second fluid medium from the fluid reservoir to the interior lumen and displacing (removing) at least a portion of the first fluid medium from the interior lumen through the fluid outlet occurs after emitting ultrasound energy from the ultrasound transducer into the region of interest.

In a further embodiment of the method, the imaging catheter and the fluid reservoir are separate components enclosed within the packaging. Additionally, the packaging can further enclose a fluid conduit extending between the fluid reservoir and the catheter to provide fluid communication between the fluid reservoir and the interior lumen such that the fluid conduit is in fluid communication with the fluid reservoir and the interior lumen as these items are removed from the packaging.

In a further embodiment of the method, the packaging further encloses a valve positioned between the fluid reservoir and the interior lumen (e.g., along the fluid conduit) such that the valve is in fluid communication with the fluid reservoir and the interior lumen of the catheter as these items are removed from the packaging. In such an embodiment, the method can further include, after removing the items from the packaging and prior to delivering at least a portion of the second fluid medium from the fluid reservoir to the interior lumen, actuating the valve from a closed position (e.g., that restricts fluid communication between the fluid reservoir and the interior lumen) to an open position (e.g., that permits fluid communication between the fluid reservoir and the interior lumen).

In a further embodiment of the method, the first fluid medium and the second fluid medium are a same type of fluid medium.

In a further embodiment of the method, at least one of the first fluid medium and the second fluid medium includes an acoustic coupling fluid medium selected from the group consisting of: castor oil, refined vegetable oil (such as canola oil), polyethylene glycol, polypropylene glycol, glycerol, and saline or other similar physiological solutions (e.g., Ringer's solution).

In a further embodiment of the method, the second fluid medium includes microbubbles and/or nanobubbles. Optionally, the microbubbles and/or the nanobubbles may further include a medication or therapeutic agent.

In a further embodiment of the method, the fluid reservoir is a syringe that comprises a plunger movable within an interior volume of the syringe. In such an embodiment, at least a portion of the second fluid medium is delivered from the fluid reservoir to the interior lumen of the imaging catheter and at least a portion of the first fluid medium is displaced from the interior lumen through the fluid outlet by moving the plunger (e.g., advancing the plunger) within the interior volume of the syringe.

Another exemplary embodiment includes a packaging assembly. This packaging assembly embodiment includes a packaging, an imaging catheter, and a fluid reservoir. The packaging defines an interior volume. The catheter is enclosed within the interior volume of the packaging. The catheter includes a catheter housing defining an interior lumen, an energy source (e.g., an ultrasound transducer, light source, etc.) disposed within the interior lumen, and a first fluid medium within the interior lumen. The fluid reservoir is also enclosed within the interior volume of the packaging. The fluid reservoir includes a second fluid medium and is in fluid communication with the interior lumen.

In a further embodiment of the packaging assembly, the catheter housing defines a fluid outlet in fluid communication with the interior lumen, and the fluid outlet is closed by a removable plug.

In a further embodiment of the packaging assembly, the imaging catheter and the fluid reservoir are separate components enclosed within the interior volume of the packaging. The packaging assembly may further include a fluid conduit enclosed within the interior volume of the packaging. The fluid conduit may extend between the fluid reservoir and the catheter to provide fluid communication between the fluid reservoir and the interior lumen of the catheter.

In a further embodiment, the packaging assembly also includes a valve enclosed within the interior volume of the packaging. The valve is positioned between the fluid reservoir and the interior lumen. The valve has a closed position that restricts fluid communication between the fluid reservoir and the interior lumen of the imaging catheter and an open position that permits fluid communication between the fluid reservoir and said interior lumen.

In a further embodiment of the assembly, at least one of the first fluid medium and the second fluid medium includes an acoustic coupling fluid medium selected from the group consisting of: castor oil, refined vegetable oil (such as canola oil), polyethylene glycol, polypropylene glycol, glycerol, and saline or other similar physiological solutions (e.g., Ringer's solution).

In some embodiments, the first fluid medium within the interior lumen of the imaging catheter may comprise a somewhat less corrosive fluid than the second fluid medium contained in the fluid reservoir. This may provide an advantage during storage, for example, since the first fluid medium may be stored for a relatively long period of time prior to use. During this storage period, for example, the first fluid medium would remain in contact with the interior lumen of the catheter; in certain embodiments, this would mean that the first fluid medium would also remain in contact with a transducer disposed within the interior volume of the catheter as well. A corrosive environment might, for example, be detrimental to a component such as a transducer. In some embodiments, a second fluid medium may be somewhat more corrosive than the first fluid medium, since the second fluid medium would be used in an acute manner (e.g., during or immediately prior to use in an imaging procedure) where the corrosion concerns would be negligible. One example of this would be a second fluid medium comprising a saline solution for flushing the catheter during an imaging procedure.

In a further embodiment of the packaging assembly, the first fluid medium includes an acoustic coupling fluid medium having an acoustic impedance within a range of about 1.0 to 3.0 MRayl. The “Rayl” or “Rayleigh” is a unit of measuring acoustic impedance. The acoustic impedance of normal blood is about 1.66 MRayls, while that of water is about 1.48 MRayl. In acoustic imaging applications, it is preferable to match the acoustic impedance to optimize acoustic coupling.

The details of one or more examples are set forth in the accompanying drawings and the description below. Other features, objects, and advantages will be apparent from the description and drawings, as well as from the claims.

### BRIEF DESCRIPTION OF DRAWINGS

The following drawings are illustrative of particular examples of the present invention and therefore do not limit the scope of the invention. The drawings are not to scale (unless so stated) and are intended for use in conjunction with the explanations in the following detailed description where like numerals in the drawings denote like elements. Examples of the present invention will hereinafter be described in conjunction with the appended drawings.

FIG. 1 is a schematic diagram of an embodiment of a system configured to perform intravascular imaging.

FIG. 2 is a schematic diagram of an embodiment of a packaging assembly for an imaging catheter and a fluid reservoir according to some embodiments.

FIG. 3 is a schematic diagram of an embodiment of an imaging catheter and fluid reservoir after being removed from the packaging assembly of FIG. 2.

FIG. 4 is a schematic diagram of an embodiment of a packaging assembly for an imaging catheter, showing a catheter in fluid communication with a plurality of fluid reservoirs within the packaging assembly.

FIG. 5 is a flow diagram of an exemplary embodiment of a method of performing an imaging procedure using an imaging catheter according to some embodiments of this disclosure.

FIG. 6 is a flow diagram of another exemplary embodiment of a method of performing an imaging procedure using an imaging catheter according to some embodiments of this disclosure.

### DETAILED DESCRIPTION

The following detailed description is exemplary in nature and is not intended to limit the scope, applicability, or configuration of the invention in any way. Rather, the following description provides some practical illustrations for implementing examples of the present invention. Those skilled in the art will recognize that many of the noted examples have a variety of suitable alternatives.

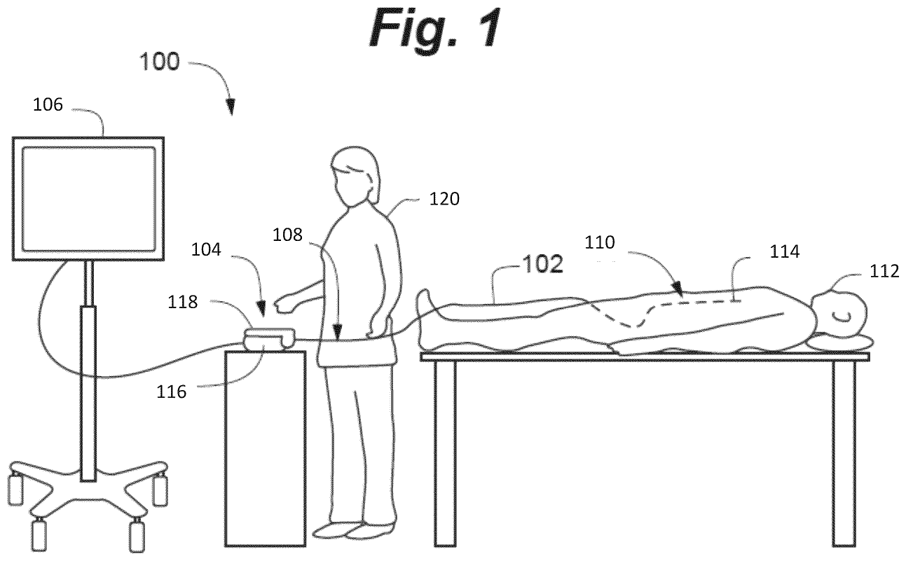

FIG. 1 illustrates an example of a system 100 that can be configured to perform intravascular imaging. The system 100 can include an imaging catheter assembly 102, a translation device 104, and an imaging engine 106. The imaging catheter assembly 102 may include a proximal end 108 and a distal end 110 that is configured to be inserted into a vessel of a patient 112. In one example, the imaging catheter assembly 102 may be inserted into the patient 112 via the femoral artery and guided to an area of interest within the patient 112. The broken lines in FIG. 1 represent portions of imaging catheter assembly 102 within the patient 112.

The imaging catheter assembly 102 can include an intravascular imaging device 114 that is configured to generate image data. The intravascular imaging device 114 can be in communication with the imaging engine 106. In some embodiments, the intravascular imaging device 114 is an ultrasound transducer configured to emit and receive ultrasound energy and, thereby, generate ultrasound image data. For instance, the image data generated by the imaging device 114 can represent a cross-section of an area of interest within the patient 112 at the location of the imaging device 114. The image data generally will represent a plurality of image items at the cross-sectional location of the imaging device 114, such as, for example, various layers of a vessel of the patient 112 and/or any accumulated matter (e.g., plaque) within the vessel of the patient 112. For example, the imaging device 114, such as an ultrasound transducer, can be configured to rotate (e.g., three hundred and sixty degrees) about a longitudinal axis of the vessel to generate image data representing the cross-section of the area of interest within the patient 112.

The translation device 104 can be configured to translate (e.g., move) the intravascular imaging device 114 of imaging catheter assembly 102 along the longitudinal axis of the vessel. The translation device 104 may comprise a linear translation system (LTS) 116. The LTS 116 may be mechanically engaged with imaging catheter assembly 102 and configured to translate imaging device 114 in a controlled manner (e.g., with respect to speed and/or distance) along the longitudinal axis of the vessel within the patient 112 during a translation operation, for example a pullback or push-forward operation. The imaging device 114, such as the ultrasound transducer, can thus rotate and translate within the vessel of the patient 112. The system 100 may comprise a patient interface module (PIM) 118 configured to interface the translation device 104 with the catheter assembly 102. Translating the imaging device 114 can allow for cross-sectional image data to be collected at various longitudinal locations within the vessel of the patient 112. This cross-sectional image data at various longitudinal locations can then be compiled, in some applications, to generate a longitudinal cross-sectional image of an area of interest.

The imaging engine 106 can be in communication with intravascular imaging device 114 and/or translation device 104. According to some examples, the imaging engine 106 may comprise at least one programmable processor. In some examples, the imaging engine 106 may comprise a computing machine including one or more processors configured to receive commands from a system user 120 and/or display image data acquired from imaging catheter assembly 102 via a user interface thereof. The computing machine may include computer peripherals (e.g., keyboard, mouse, electronic display) to receive inputs from the system user 120 and output system information and/or signals received from imaging catheter assembly 102 (e.g., rendered images). In some examples, the user interface of the computing machine may be a touch screen display configured to act as both an input device and an output device. In some examples, imaging engine 106 may include memory modules for storing instructions, or software, executable by the one or more processors.

Having described an exemplary intravascular imaging system and the related generation of image data, this disclosure will now describe details related to embodiments of the imaging catheter assembly used in such system to generate image data as well as related packaging assemblies and methods of using the catheter.

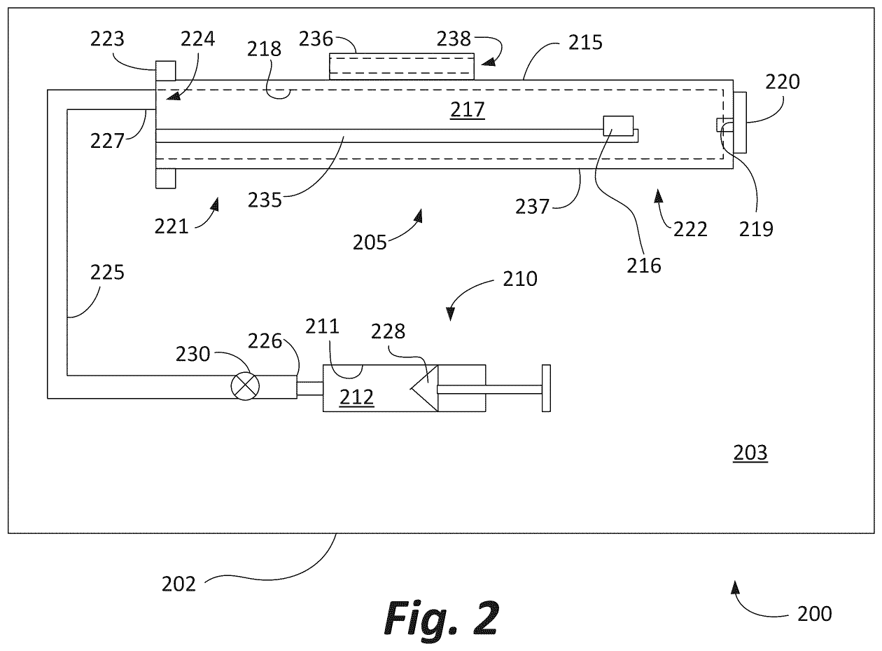

FIG. 2 illustrates a schematic diagram of an embodiment of a packaging assembly 200. In particular, FIG. 2 shows a schematic, top plan view of the packaging assembly 200.

The illustrated packaging assembly 200 includes a packaging 202. The packaging 202 defines an interior volume 203. Disposed within the interior volume 203 of the packaging 202 is an imaging catheter 205 (e.g., an imaging catheter assembly) and a fluid reservoir 210. The packaging 202 can be in the form of a packaging container that, for instance, seals the catheter 205 and fluid reservoir 210 (as well as any other components) enclosed within the interior volume 203 of the packaging 202. This can maintain the contents within the interior volume 203 of the packaging 202 in a sterile state by preventing ingress of the ambient environment into the interior volume 203 until the packaging 202 is opened and the catheter 205 and fluid reservoir 210 are removed from the packaging 202, for example, just prior to use in an imaging procedure. In the illustrated example, the catheter 205 and the fluid reservoir 210 can be fully assembled when enclosed within the packaging 202. In this way, once the catheter 205 and the fluid reservoir 210 are removed from the packaging 202, the catheter 205 may be operably coupled to the imaging engine 106 (e.g., via intermediary components and connections, as applicable) and positioned within a patient's blood vessel to begin imaging. In another example, certain components can be fully assembled when enclosed within the packaging 202, while one or more other components can be in an unassembled state (either enclosed within the packaging 202 or separate from the packaging 202).

It should be noted that the catheter 205 depicted in the accompanying figures (such as FIG. 2) is greatly simplified in order to show various details. For example, catheter 205 may have a total length of more than 50 cm, or more than 100 cm, or more than about 130 cm in some embodiments. In certain exemplary embodiments, catheter 205 may have a total length of between 135 cm and 150 cm. As such, catheter 205 is often coiled in a generally circular manner (not shown in the figures) to facilitate placement within packaging 202 during manufacturing and packaging, for example. Commonly, a catheter dispensing hoop or a dispensing coil arrangement (not shown) may be included within packaging 202 to house and/or constrain catheter 205 during storage, and to later facilitate dispensing and placement of catheter 205 following removal from packaging 202 for use during an imaging procedure.

The catheter 205 can include a catheter housing 215, an imaging device 216, and a first fluid medium 217. In the illustrated embodiment, the imaging device 216 can be an ultrasound transducer configured to transmit and/or receive ultrasound energy, for example. The catheter housing 215 can define an interior lumen 218. Each of the imaging device 216 (e.g., the ultrasound transducer) and the first fluid medium 217 can be disposed within the interior lumen 218. The first fluid medium 217 contained within the interior lumen 218 can surround, and thereby contact, the imaging device 216. Accordingly, in the illustrated example, when the catheter 205 is used to generate image data (e.g., after removing the catheter 205 from the packaging 202 and insertion into a patient's blood vessel), the first fluid medium 217 can provide an acoustic coupling medium through which ultrasound energy can be conveyed.

The catheter housing 215 can also define a fluid outlet 219. The fluid outlet 219 may be an opening in a distal portion 222 of catheter 205, and can be in fluid communication with the interior lumen 218. In one example, as shown in FIG. 2, the fluid outlet 219 can be closed when the catheter 205 is within the packaging 202. For example, the fluid outlet 219 may be sealed or closed by a removable fluid barrier, such as a removable plug 220. In an optional embodiment, a removable plug 220 may be formed as part of a catheter dispensing hoop or dispensing coil arrangement (not shown), as are commonly used in the packaging of catheter assemblies. Such a removable plug 220 could be attached to, or formed integrally with, the hoop or coil arrangement, for example. In such an embodiment, the removable plug 220 would be removed from fluid outlet 219 in conjunction with removing the dispensing hoop or coil from catheter 205 just prior to use. In other embodiments, fluid outlet 219 may be created or formed in a distal portion 222 of catheter housing 215 by puncturing or piercing, or by removing a detachable portion, or by employing similar methods.

In some embodiments, the catheter housing 215 can define a proximal portion 221 generally at or near one end, and a distal portion 222 generally at or near an opposite longitudinal end of the catheter housing 215. When the catheter 205 is in use during an imaging procedure, as shown in FIG. 1, the proximal portion 221 can generally be positioned outside of a patient while the distal portion 222 can generally be positioned inside of the patient (e.g., within a vessel of the patient). The interior lumen 218 can be defined by the housing 215 and extends generally from the proximal portion 221 to the distal portion 222. In one example, a volume of the interior lumen 218 which extends from the proximal portion 221 to the distal portion 222 is substantially filled with the first fluid medium 217. In one embodiment, the volume of the interior lumen 218 can fall within a range of volumes, such as 0.5-5 milliliters, 0.75-4 milliliters, 1-3 milliliters, or 1-2 milliliters. In some embodiments, the interior lumen 218 may be substantially filled with the first fluid medium 217.

The proximal portion 221 can include a catheter hub 223. The hub 223 may, in some instances, define a port 224. The port 224 can be in fluid communication with the interior lumen 218 and, accordingly, be located at any location on the hub 223 where it is in fluid communication with the interior lumen 218. The port 224 may be adapted, for instance, to fluidly couple to a device, such as a vacuum device (not shown). As such, the port 224 can include one or more structures configured to couple (e.g., in a fluid tight or sealed manner) to the vacuum device. In one example, the port 224 can take the form of a luer connection component, for instance where the port 224 is a female luer component and the vacuum device has a male luer component adapted for fluidly coupling the vacuum device to port 224. In another example, the port 224 can be threaded to receive corresponding threading of the vacuum device. Because the port 224 is in fluid communication with the interior lumen 218, coupling the vacuum device to the port 224 brings the vacuum device into fluid communication with the interior lumen 218. In some instances, the interior lumen 218 can be filled with the first fluid medium 217 using the vacuum device. In one such instance, the first fluid medium 217 can be introduced into the interior lumen 218 through the same port 224 to which the vacuum device is coupled. This may include introducing the first fluid medium into the interior lumen 218 through the vacuum device itself and can include degassing the first fluid medium, for example. In another instance, the first fluid medium can be introduced into the interior lumen 218 through a separate fluid opening in the catheter hub 223 if and when present. In some embodiments, a vacuum device may be connected to port 219 to help facilitate filling of the interior lumen 218 with the first fluid medium 217.

The distal portion 222 can include the imaging device 216. In the illustrated example, the imaging device 216 is an ultrasound transducer. The ultrasound transducer can be positioned within the interior lumen 218, such as at a distal portion of the interior lumen 218 as shown. The imaging device 216 (e.g., an ultrasound transducer) can be coupled to a drive cable 235 also located within the interior lumen 218. The drive cable 235 can extend within the interior lumen 218, and the drive cable 235 can be used to change the rotational position and/or the longitudinal position of the imaging device 216 within the catheter housing 215 during image data generation. The catheter 205 may be further adapted to receive a guidewire at a guidewire receiver 236, typically disposed near the distal portion 222 of the catheter 205, to guide the distal portion 222 (and the accompanying catheter 205) to a region of interest within the vessel of the patient and/or translate the distal portion 222 within the vessel, such as during imaging. When the guidewire receiver 236 is included, the guidewire receiver 236 may be disposed along a surface of catheter housing 215 and may extend outwardly from the housing 215 (e.g., disposed on an outer surface). In some embodiments, guidewire receiver 236 may be disposed proximate the distal portion 222 of catheter housing 215. The guidewire receiver 236 may define a guidewire lumen 238 through which a guidewire is received. The guidewire, when so employed, facilitates guiding the distal portion 222 of the housing 215 as it is traversed through a vessel to an area of interest. In other embodiments, the distal portion 222 may be directly guided into the vessel without the use of the guidewire.

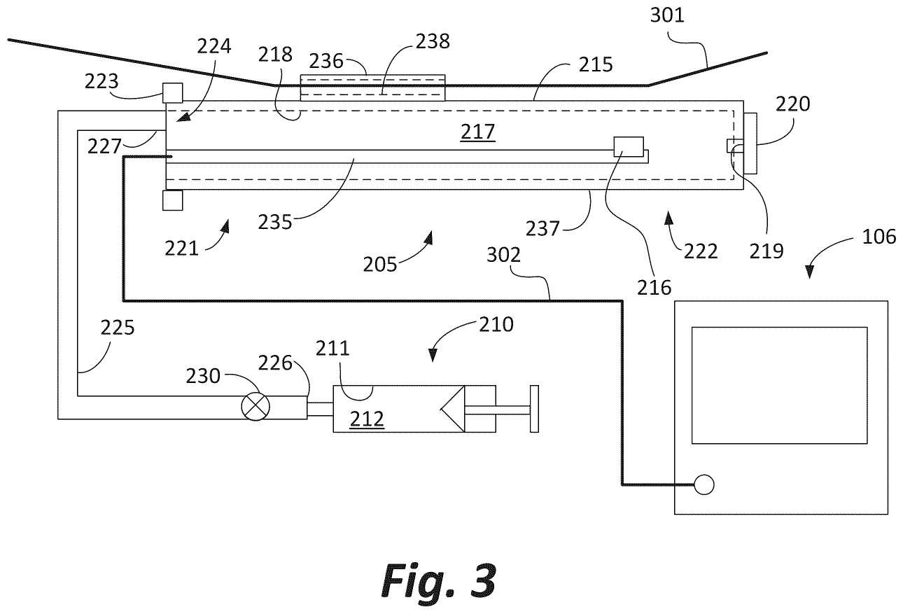

FIG. 3 is a schematic diagram of an embodiment of an imaging catheter 205 and a fluid reservoir 210 after being removed from the packaging assembly 200 of FIG. 2 in anticipation of use during an imaging procedure. For example, in FIG. 3, a fluid conduit 225 is shown providing fluid communication between fluid reservoir 210 and interior lumen 218 of imaging catheter 205. In the embodiment depicted in FIG. 3, guidewire receiver 236 is shown disposed along a surface of catheter housing 215. In detail, guidewire receiver 236 is shown extending outwardly from an outer surface of housing 215. Further, guidewire receiver 236 may be disposed near a distal portion 222 of catheter housing 215. Guidewire 301 is also shown in FIG. 3 being received by guidewire lumen 238 in guidewire receiver 236. The guidewire 301, when so employed, facilitates guiding the distal portion 222 of catheter 205 as it is traversed through a vessel to an area of interest. FIG. 3 also shows schematically a simplified connection 302 between device cable 235 of catheter 205 and imaging engine 106. Connection 302 may provide electrical signal pathways used, for example, to transmit electrical energy in a first direction to the ultrasound transducer 216 to thereby generate and transmit ultrasound energy from ultrasound transducer 216 in some embodiments, and further to transmit electrical signals converted from reflected ultrasound energy received by transducer 216 in a second direction to imaging engine 106 for processing, display, manipulation, storage, etc. In some embodiments, connection 302 may also represent the means for moving/driving (e.g., rotating and/or translating longitudinally) the drive cable 235 during imaging procedures. As such, connection 302 may represent a number of connected components that were described above with respect to FIG. 1 and depicted therein. For example, connection 302 may comprise a translation device 104, which may further comprise a linear translation system (LTS) 116, in some embodiments. Additionally, connection 302 may comprise a patient interface module (PIM) 118, configured to interface the translation device 104 with the catheter assembly 102.

With reference again to FIG. 2, an imaging window 237 may be located at the distal portion 222 of the housing 215. The imaging window 237 may comprise a portion of the catheter housing 215 and may be composed of a material that is substantially transparent to ultrasound energy, for example, energy emitted by ultrasound transducer 216 in certain embodiments. In many cases, the imaging window 237 extends circumferentially around a perimeter (e.g., a circumference) of the catheter housing 215, and may extend longitudinally over a length of the catheter housing 215 near the distal portion 222. As one example, the imaging window 237 can be made up of a number of segments fused together at the respective interfaces so as to be in direct contact and form a continuous, fluid impermeable segment of the catheter housing 215 that is transparent to ultrasound energy (e.g., without the presence of any adhesive material between the imaging window segments). Two or more (e.g., all) of these fused imaging window segments can be of differing flexural moduli (e.g., each having a different flexural modulus) so as to allow the catheter housing 215 to effectively traverse a vessel while at the same time being controllable from the opposite proximal portion. An imaging window 237 having varying flexural moduli along its length is described, for instance, in U.S. Pub. Pat. App. No. 2017/0055943, the contents of which are incorporated by reference in relevant part.

As noted previously, one or more fluid reservoirs 210 may also be disposed within interior volume 203 of packaging 202. The fluid reservoir 210 can include a second fluid medium 212. For instance, the fluid reservoir 210 can define a reservoir interior volume 211 within which the second fluid medium 212 is included. In some embodiments, the fluid reservoir 210 can be configured to be in fluid communication with the interior lumen 218 of catheter 205 such that the second fluid medium 212 can be delivered from the fluid reservoir 210 to the catheter 205 (e.g., to the interior lumen 218 of the catheter 205). As shown here, the catheter 205 and the fluid reservoir 210 can be separate components enclosed within the interior volume 203 of the packaging 202. As one example, the fluid reservoir 210 can be a syringe (e.g., a hand-held syringe) that includes a plunger 228. The plunger 228 can be movable within the interior volume 211 of the syringe (e.g., by moving an actuator coupled to plunger 228). As a result of moving the plunger 228 toward the outlet of the fluid reservoir 210 (and, thereby, increasing the pressure of the second fluid medium 212 within the fluid reservoir 210), at least a portion of the second fluid medium 212 can be delivered from the fluid reservoir 210 to the interior lumen 218 of catheter 205. In some embodiments, as a result of moving the plunger 228 within the interior volume 211 and, thereby, delivering the second fluid medium 212 to the interior lumen 218, at least a portion of the first fluid medium 217 can be displaced from the interior lumen 218 through the fluid outlet 219. The foregoing description assumes that fluid outlet 219 is not obstructed by the presence of, for example, plug 220.

In the exemplary embodiment illustrated in FIG. 2, the fluid reservoir 210 can be in fluid communication with the interior lumen 218 while the catheter 205 and the fluid reservoir 210 are enclosed within the packaging 202. For example, the packaging assembly 200 can further include a fluid conduit 225. The fluid conduit 225, like the catheter 205 and the fluid reservoir 210, can be enclosed within the interior volume 203 of the packaging 202. While enclosed within the packaging 202, the fluid conduit 225 can extend between the fluid reservoir 210 and the catheter 205 to provide fluid communication between the fluid reservoir 210 and the interior lumen 218 of catheter housing 215. For example, the fluid conduit 225 can have a first end portion 226 fluidly connected to the fluid reservoir 210 and a second, opposite end portion 227 fluidly connected to the catheter 205 (e.g., fluidly connected to the interior lumen 218). When the packaging 202 is opened (e.g., in anticipation of use of the catheter 205 in a procedure), the catheter 205, the fluid reservoir 210, and the fluid conduit 225 can be removed from the packaging 202 with the fluid conduit 225 already in fluid communication with each of the fluid reservoir 210 and the interior lumen 218, according to some embodiments.

The illustrated embodiment also shows the exemplary packaging assembly 200 including an optional valve 230. The valve 230 can be enclosed within the interior volume 203 of the packaging 202. More specifically, the valve 230 can be positioned between the fluid reservoir 210 and the interior lumen 218. In this way, the valve 230 can be in fluid communication with both the interior lumen 218 of the catheter 205 and the fluid reservoir 210 while the valve 230, the catheter 205, and the fluid reservoir 210 are each enclosed within the packaging 202. When the packaging 202 is opened, the valve 230 can be removed from the packaging 202 while being in fluid communication with each of the fluid reservoir 210 and the interior lumen 218. The illustrated example shows the valve 230 being formed integrally with the fluid conduit 225 and positioned near the fluid reservoir 210. However, in other examples the valve 230 can be positioned at other locations, such as near the inlet of the interior lumen 218 of the catheter 205. The valve 230 can have a closed position and an open position. In the closed position, the valve 230 can restrict or prevent fluid communication between the fluid reservoir 210 and the interior lumen 218. In the open position, the valve 230 can permit fluid communication between the fluid reservoir 210 and the interior lumen 218.

As described previously, the catheter 205 can include a first fluid medium 217 within the interior lumen 218 and the fluid reservoir 210 can include a second fluid medium 212. Depending on the application in which the packaging assembly 200 is to be used, the first fluid medium 217 and the second fluid medium 212 can be the same or the same type of fluid medium, or they may be different types of fluid medium. In some embodiments, at least one of the first fluid medium 217 and the second fluid medium 212 can include an acoustic coupling fluid medium. In some such embodiments, the first fluid medium 217 and the second fluid medium 212 can each be an acoustic coupling fluid medium type. For example, in such embodiments, at least one of the first fluid medium 217 and the second fluid medium 212 can include a glycol-type acoustic coupling fluid medium, or an acoustic coupling fluid comprising castor oil or refined vegetable oil (such as canola oil) in some embodiments. More specifically, in such embodiments, at least one of the first fluid medium 217 and the second fluid medium 212 can include an acoustic coupling fluid medium selected from the group consisting of: castor oil, refined vegetable oil (such as canola oil), polyethylene glycol, polypropylene glycol, glycerol, and saline or other similar physiological solutions (e.g., Ringer's solution). Where the first fluid medium 217 and/or the second fluid medium 212 is an acoustic coupling fluid medium, the first fluid medium 217 and/or the second fluid medium 212 may have an acoustic impedance value that falls within a range of values, for example, a range of 0.5-5.0 MRayl, 0.75-4.0 MRayl, 1.0-3.0 MRayl, or 1.25-2.5 MRayl. In some embodiments, the first fluid medium 217 and/or the second fluid medium 212 may be an acoustic coupling fluid medium having an acoustic impedance value of about 1.66 MRayl to provide appropriate acoustic coupling or acoustic matching in an environment comprising typical blood, for example.

For example, where the packaging assembly 200 is to be used in an application where the fluid reservoir 210 could be useful to flush the interior lumen 218, which is pre-filled with the first fluid medium 217 while within the packaging 202, the second fluid medium 212 within the fluid reservoir 210 can be the same, or a similar type of acoustic coupling fluid medium as the first fluid medium 217. In this way, the catheter 205 and fluid reservoir 210 can be removed from the packaging 202 and be ready for use. Namely, in one example, upon removal from the packaging 202, the catheter 205 can be ready for use in collecting image data with the first fluid medium 217 serving as an acoustic coupling fluid around the ultrasound transducer and, after collecting an initial set of image data, the second fluid medium 212 can be delivered from the fluid reservoir 210 to the interior lumen 218 to serve as an acoustic coupling fluid medium for the ultrasound energy emitted from, and received at, the ultrasound transducer. Delivering the second fluid medium 212 after collecting the initial set of image data can be useful in removing or displacing (e.g., via the fluid outlet 219) any air bubbles that may have formed (e.g., as a result of translational and/or rotational movement of the ultrasound transducer) during the initial set of image data. In another example, where air bubbles may have formed within the interior lumen 218 while the catheter 205 was within the packaging 202 (during storage, for example), upon removal from the packaging 202, the second fluid medium 212 can be delivered from the fluid reservoir 210 to the interior lumen 218 to both remove those air bubbles prior to imaging (or after initial image associated with setup) and then serve as an acoustic coupling fluid medium for the ultrasound energy emitted from, and received at, the ultrasound transducer 216 during the imaging. In one further such example, after the initial delivery of the second fluid medium 212 to the interior lumen 218 and collecting some image data, additional second fluid medium 212 can be delivered from the fluid reservoir 210 to the interior lumen 218 to remove any air bubbles that may have formed during that image data collection, and so forth.

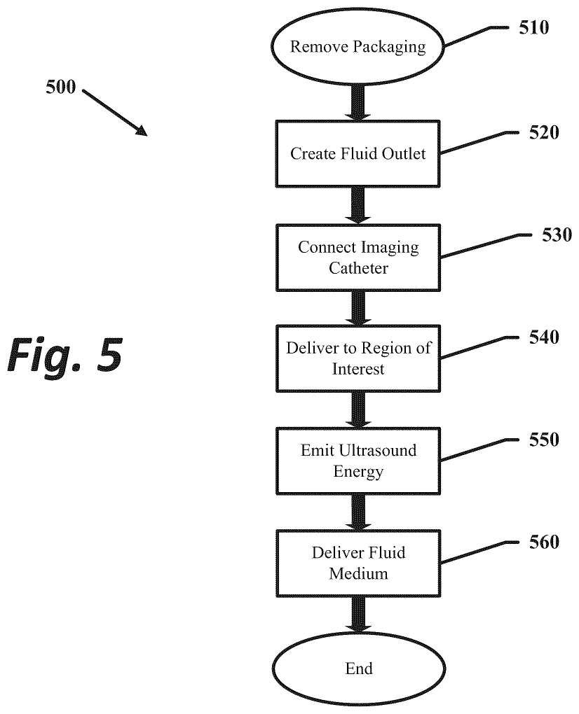

FIG. 5 is a flowchart describing a method 500 of using a packaging assembly containing an imaging catheter and a fluid reservoir to perform an imaging procedure according to some embodiments of this disclosure. In the following description, reference is also made to corresponding reference numerals from FIGS. 2 and 3. For example, FIG. 5 shows a number of steps that may be performed during an imaging procedure using an imaging catheter and a fluid reservoir as described in this disclosure. Step 510 of method 500 comprises removing packaging 202 from a packaging assembly 200 having an imaging catheter 205 and a fluid reservoir 210 disposed within an interior volume 203 of packaging 202. Steps 520 and 530 of method 500 are shown next, although the order of performing these two steps is not strictly required; step 530 could be performed prior to performing step 520, for example. Step 520 comprises creating a fluid outlet 219 in the catheter housing 215. Creating fluid outlet 219 can be accomplished in several ways, as previously described herein, including by removing a plug 220 from catheter housing 215. Step 530 comprises connecting the imaging catheter 205 to an imaging engine 106. As noted above, connecting the imaging catheter 205 to imaging engine 106 may involve a number of intermediate connected components such as a translation device 104, which may further comprise a linear translation system (LTS) 116, and a patient interface module (PIM) 118 configured to interface (e.g., connect) the translation device 104 with the catheter assembly 102. Step 540 comprises delivering imaging catheter 205 to a region of interest (e.g., a vessel within a patient). This step may be performed by a physician, for example, using x-ray fluoroscopy to navigate the imaging catheter 205 to a region of interest. Step 550 comprises emitting ultrasound energy from the ultrasound transducer 216 of the imaging catheter 205 at or near the region of interest. It should be noted here that the order of performing steps 540 and then 550 is not strictly required; step 550 could be performed prior to performing step 540 according to some embodiments. For example, the ultrasound transducer 216 may be turned on (and ultrasound energy emitted therefrom) to evaluate its functioning and/or imaging capability prior to delivering the imaging catheter 205 to a region of interest in the patient. Step 560 comprises delivering a second fluid medium 212 from fluid reservoir 210 to an interior lumen 218 of catheter 205 such that it displaces at least some of the first fluid medium 217 in lumen 218 through outlet 219. It should be noted that steps 550 and 560 are not necessarily performed in this order, and that in some circumstances, it may be desirable to deliver the second fluid medium 212 to the interior lumen 218 before emitting ultrasound energy. It may also be desirable in some circumstances to deliver the second fluid medium 212 to the interior lumen 218 before delivering the imaging catheter 205 to the region of interest.

In some embodiments, the fluid reservoir 210 could be used to facilitate delivering a therapy, or in conjunction with therapy delivery. This could be accomplished, for example, in embodiments where the second fluid medium 212 is different from the first fluid medium 217. The second fluid medium 212 may comprise a therapeutic or medicinal fluid medium, for example. The second fluid medium 212 may, for example, include “microbubbles” that may possess therapeutic properties. Such microbubbles may be present in certain ultrasound contrast agents (for example, those used in contrast enhanced ultrasonography), or may include therapeutic agents (e.g., a medication). In this context, microbubbles in second fluid medium 212 may comprise suspensions of gas bubbles of nano- and/or micro-metric size dispersed in an aqueous medium. The gas may be entrapped or encapsulated in a film-layer comprising, for instance, emulsifiers, oils, thickeners or sugars. Such microbubbles are sometimes also referred to in the art by various other names, such as “microspheres,” “microcapsules,” “microballoons,” and “microvesicles.” Methods of producing certain ultrasound contrast agents containing microbubbles are disclosed, for example, in international published patent applications WO1994/009829 and in WO2004/069284, the respective contents of which are hereby incorporated by reference in relevant part. Examples of commercially available ultrasound contrast agents that may potentially be useful as a second fluid medium 212 include, but are not limited to, SonoVue®, Lumason®, Definity, and Optison.

In some embodiments, imaging catheter 205 may be used to generate image data of a region of interest. The image data of the region of interest can then be used to inform a decision to deliver a therapy, such as to determine whether a therapeutic or medicinal fluid medium (e.g., microbubbles including a medication) from fluid reservoir 210 should be delivered and thereby applied to the region of interest, or possibly to determine an appropriate type of therapeutic or medicinal fluid medium to be delivered from a fluid reservoir 210. Upon determining that the therapy should be applied, the therapeutic or medicinal fluid medium from the fluid reservoir 210 can be delivered to the region of interest.

In other exemplary cases, the fluid reservoir can include a fluid medium that serves as a cooling fluid for use with an ablation procedure. The catheter 205 could, for example, be configured to deliver energy (e.g., via the ultrasound transducer) to a region of interest to provide a form of ablation or thrombolysis effect (e.g., to break up or dissolve a thrombus), and the fluid medium from the fluid reservoir 210 could subsequently be delivered to cool one or more areas associated with such an ablation procedure (e.g., to cool the region of interest following such an energy delivery, etc.). The second fluid medium 212 could, for example, comprise a cooling fluid for use in conjunction with an ablation procedure. The first fluid medium 217 in the interior lumen 218 of catheter 205 could, for example, be used to deliver ablation energy (e.g., via the ultrasound transducer) to a region of interest, and the second fluid medium 212 in the fluid reservoir 210 could be delivered to cool one or more areas associated with the ablation procedure (e.g., the vessel lumen, region of interest, etc.).

As another particular example, catheter 205 could be used to provide a catheter-based, ultrasound thrombolysis or ultrasound accelerated thrombolysis. In this example, the therapeutic or medicinal fluid medium delivered by fluid reservoir 210 may comprise a thrombolytic drug, such as a tissue plasminogen activator (“tPA”). A tPA solution could be delivered to the vessel or region of interest via first fluid medium 217 within interior lumen 218, or via one or more fluid reservoirs 210, through the fluid outlet 219 of catheter 205 to the region of interest, for example. In conjunction with the delivery of the tPA solution, ultrasound energy could be generated by ultrasound transducer 216 to thereby enhance or accelerate the thrombolytic effect of the tPA. Additionally or alternatively, the tPA could be integrated in a microbubble form as described above, wherein the microbubbles in a fluid medium could be delivered to target a thrombus in a vessel at a region of interest, for example, and ultrasound energy delivery may cause a release of the tPA from the microbubbles to locally deliver tPA to a thrombus. The localized delivery of tPA in this manner may provide a therapeutic benefit at the region of interest (e.g., to the thrombus) without significantly increasing systemic tPA concentrations in a patient, which may be undesirable for some patients (e.g., may be a risk factor for hemorrhagic stroke, for example). Further, a subsequent delivery of a cooling fluid (e.g., second fluid medium 212 from one or more of the fluid reservoirs 210) could be delivered to cool one or more areas associated with the above-described tPA ultrasound accelerated thrombolysis procedure.

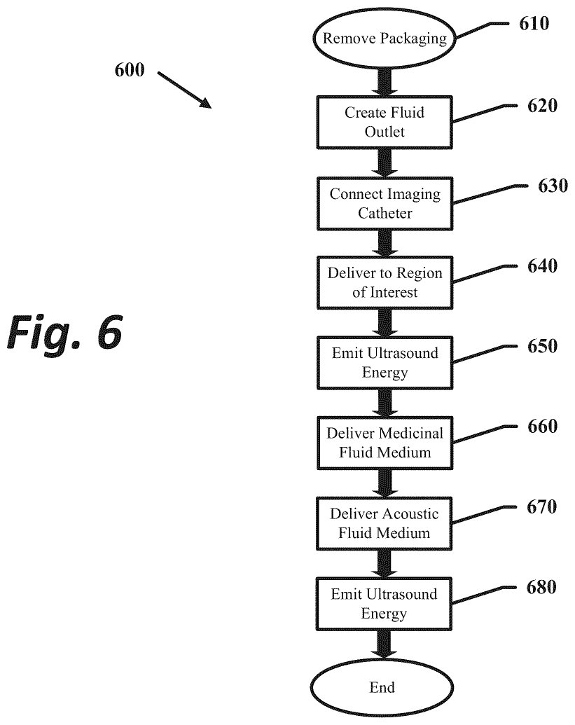

FIG. 6 is a flowchart describing a method 600 of using a packaging assembly containing an imaging catheter and a fluid reservoir to perform an imaging procedure and to deliver a therapeutic or medicinal fluid medium according to some embodiments of this disclosure. In the following description, reference is also made to corresponding reference numerals from FIGS. 2 and 3. For example, FIG. 6 shows a number of steps that may be performed during an imaging procedure using a catheter and fluid reservoir as described in this disclosure. Step 610 of method 600 comprises removing packaging 202 from a packaging assembly 200 having an imaging catheter 205 and a fluid reservoir 210 disposed within an interior volume 203 of packaging 202. Steps 620 and 630 of method 600 are shown next, although the order of performing these two steps is not strictly required; step 630 could be performed prior to performing step 620, for example. Step 620 comprises creating a fluid outlet 219 in the catheter housing 215. Creating fluid outlet 219 can be accomplished in several ways, as previously described herein, including by removing a plug 220 from catheter housing 215. Step 630 comprises connecting the imaging catheter 205 to an imaging engine 106. As noted above, connecting the imaging catheter 205 to imaging engine 106 may involve a number of intermediate connected components such as a translation device 104, which may further comprise a linear translation system (LTS) 116, and a patient interface module (PIM) 118 configured to interface (e.g., connect) the translation device 104 with the catheter assembly 102. Step 640 comprises delivering imaging catheter 205 to a region of interest (e.g., a vessel within a patient). This step may be performed by a physician, for example, using x-ray fluoroscopy to navigate the imaging catheter 205 to a region of interest. Step 650 comprises emitting ultrasound energy from the ultrasound transducer 216 of the imaging catheter 205 at or near the region of interest. It should be noted here that the order of performing steps 640 and then 650 is not strictly required; step 650 could be performed prior to performing step 640 according to some embodiments. For example, the ultrasound transducer 216 may be turned on (and ultrasound energy emitted therefrom) to evaluate its functioning and/or imaging capability prior to delivering the imaging catheter 205 to a region of interest in the patient. Step 660 comprises delivering a therapeutic or medicinal fluid medium 212 from fluid reservoir 210 to an interior lumen 218 of catheter 205 such that it is delivered through lumen 218, through outlet 219, and to a therapy delivery location. In some embodiments, step 660 may be performed based on the results of step 650; that is, the imaging data generated during the emission of ultrasound energy in step 650 may inform the decision of whether to deliver a therapeutic or medicinal fluid medium 212, or which type of therapeutic or medicinal fluid medium 212 to deliver, and/or possibly where to deliver the therapeutic or medicinal fluid medium 212, for example. Step 670 comprises delivering an acoustic fluid medium from fluid reservoir 210 to an interior lumen 218 of catheter 205. Step 670 may be performed prior to step 680, which comprises emitting ultrasound energy to a region of interest. Steps 670 and 680 may be performed, for example, following the delivery of a therapeutic or medicinal fluid medium in order to assess the effect of the therapeutic fluid delivery. It should be noted that steps 650, 660, 670, and 680 do not necessarily need to be performed in this order, and that in some circumstances, it may be desirable to alter the order of the steps and/or omit certain steps, depending on the circumstances presented during the procedure. For example, the above-described tPA ultrasound accelerated thrombolysis protocol would likely involve slight modifications to the order of performance of the steps of method 600, such as delivering the medicinal fluid medium (step 660) comprising tPA prior to emitting ultrasound energy (step 650), and subsequently delivering an acoustic fluid medium (step 670 in the form of a cooling medium, etc., according to some embodiments of this disclosure.

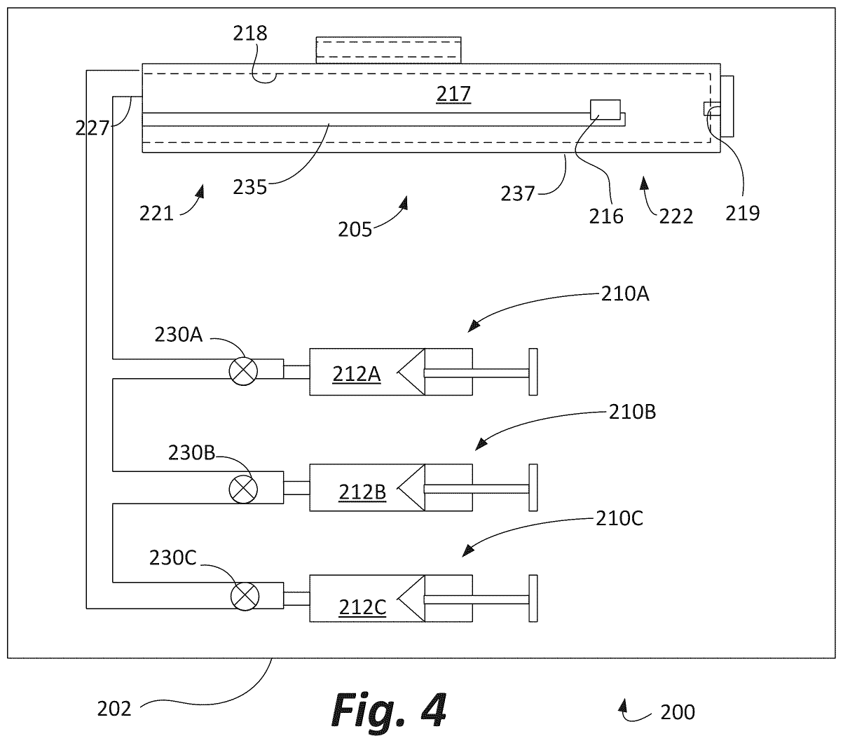

FIG. 4 is a schematic diagram of an embodiment of a packaging assembly 200 for an imaging catheter 205, showing catheter 205 in fluid communication with a plurality of fluid reservoirs (e.g., syringes 210A, 210B, and 210C in the example shown in FIG. 4) disposed within packaging 202 of the packaging assembly 200. Fluid reservoirs 210A, 210B, and 210C may possibly contain different types of fluids, indicated as second fluid media 212A, 212B, and 212C in FIG. 4. The provision of multiple fluid reservoirs (e.g., two or more) may facilitate employing different options, e.g., therapeutic options, following or in conjunction with an imaging procedure. For example, depending on the imaging data that are received and interpreted using imaging catheter 205, it may be desirable to next deliver a therapeutic agent, or a contrast agent, or a saline solution, or a fluid comprising or containing medicinal microbubbles, etc. In some cases, the imaging data received during an initial stage of an imaging procedure may be used to determine whether to perform subsequent therapeutic steps, and/or which types of therapeutic steps to perform, for example. In some embodiments, having two or more fluid reservoirs 210 within packaging 202 may allow for flushing air bubbles with an acoustic coupling fluid from a fluid reservoir prior to performing an imaging procedure, and then, if desired, delivering a therapeutic or medicinal fluid from a different fluid reservoir to deliver a certain therapy, for example. FIG. 4 shows an embodiment having three fluid reservoirs 210A, 210B, and 210C within packaging 202, but those of ordinary skill in the art would understand that the number could vary depending on the evolving clinical needs and/or other factors. Other alternative embodiments may include, for example, the ability to disconnect and fill/refill a fluid reservoir, or the provision of therapeutic or medicinal fluids in a series of steps (e.g., successive deliveries).

Various examples have been described. These and other examples are within the scope of the following claims.

What is claimed is:

1. A packaging assembly comprising:
a packaging defining an interior volume;
an imaging catheter enclosed within the interior volume of the packaging, the catheter including a catheter housing defining an interior lumen, an energy source disposed within the interior lumen, and a first fluid medium within the interior lumen; and
a fluid reservoir enclosed within the interior volume of the packaging, the fluid reservoir including a second fluid medium, the fluid reservoir being in fluid communication with the interior lumen of the catheter.

2. The assembly of claim 1, wherein the energy source comprises an ultrasound transducer.

3. The assembly of claim 1, wherein the energy source comprises a light source.

4. The assembly of claim 1, wherein the catheter housing defines a fluid outlet in fluid communication with the interior lumen, and wherein the fluid outlet is closed by a removable plug.

5. The assembly of claim 1, wherein the catheter and the fluid reservoir are separate components enclosed within the interior volume of the packaging, and wherein the assembly further comprises a fluid conduit enclosed within the interior volume of the packaging, wherein the fluid conduit extends between the fluid reservoir and the catheter to provide fluid communication between the fluid reservoir and the interior lumen of the catheter.

6. The assembly of claim 1, further comprising a valve enclosed within the interior volume of the packaging, wherein the valve is positioned between the fluid reservoir and the interior lumen, and wherein the valve has a closed position that restricts fluid communication between the fluid reservoir and the interior lumen and an open position that permits fluid communication between the fluid reservoir and the interior lumen.

7. The assembly of claim 2, wherein at least one of the first fluid medium and the second fluid medium comprises an acoustic coupling fluid medium selected from the group consisting of: castor oil, refined vegetable oil, polyethylene glycol, polypropylene glycol, glycerol, saline, and Ringer's solution.

8. The assembly of claim 2, wherein the first fluid medium comprises an acoustic coupling fluid medium having an acoustic impedance of 1.0-3.0 MRayl.

9. The assembly of claim 4, further comprising a dispensing hoop for holding the catheter within the interior volume of the packaging, wherein the removable plug comprises a portion of the dispensing hoop configured to be removably disposed in the fluid outlet.

10. The assembly of claim 9, wherein the removable plug is integrally formed in the dispensing hoop and is configured to be removed from the fluid outlet prior to use of the catheter.

11. A method of using an ultrasound imaging catheter, the method comprising the steps of:
providing a packaging assembly comprising a packaging, the imaging catheter, and a fluid reservoir, the imaging catheter and the fluid reservoir being enclosed within the packaging, wherein the imaging catheter comprises a catheter housing defining an interior lumen, an ultrasound transducer disposed within the interior lumen, and a first fluid medium within the interior lumen, and wherein the fluid reservoir comprises a second fluid medium within an interior volume of the fluid reservoir, the fluid reservoir being in fluid communication with the interior lumen of the imaging catheter;
removing the imaging catheter and the fluid reservoir from the packaging;
creating a fluid outlet in a distal portion of the catheter housing, the fluid outlet being in fluid communication with the interior lumen of the imaging catheter;
connecting the imaging catheter to an imaging engine;
delivering the imaging catheter to a region of interest within a patient;
emitting ultrasound energy from the ultrasound transducer into the region of interest; and
delivering at least a portion of the second fluid medium from the fluid reservoir to the interior lumen and displacing at least a portion of the first fluid medium from the interior lumen through the fluid outlet.

12. The method of claim 11, wherein creating the fluid outlet at the interior lumen comprises opening an aperture defined at the catheter housing.

13. The method of claim 12, wherein opening the aperture defined at the catheter housing comprises removing a plug from the aperture.

14. The method of claim 13, wherein the packaging assembly further comprises a catheter dispensing hoop having the plug integrally formed therein, and wherein removing the plug from the aperture occurs as a result of removing the imaging catheter from the dispensing hoop.

15. The method of claim 11, wherein delivering at least the portion of the second fluid medium from the fluid reservoir to the interior lumen and displacing at least the portion of the first fluid medium from the interior lumen through the fluid outlet occurs after emitting ultrasound energy from the ultrasound transducer into the region of interest.

16. The method of claim 11, wherein delivering at least the portion of the second fluid medium from the fluid reservoir to the interior lumen and displacing at least the portion of the first fluid medium from the interior lumen through the fluid outlet occurs before delivering the imaging catheter to the region of interest.

17. The method of claim 11, further comprising emitting ultrasound energy from the ultrasound transducer before delivering the imaging catheter to the region of interest within the patient.

18. The method of claim 11, wherein the catheter and the fluid reservoir are separate components enclosed within the packaging.

19. The method of claim 18, wherein the packaging assembly further comprises a fluid conduit enclosed within the packaging, the fluid conduit extending between the fluid reservoir and the interior lumen of the imaging catheter to provide fluid communication between the fluid reservoir and the imaging catheter.

20. The method of claim 19, wherein the packaging assembly further comprises a valve enclosed within the packaging, the valve being disposed along the fluid conduit between the fluid reservoir and the interior lumen.

21. The method of claim 20, further comprising, after removing the imaging catheter and the fluid reservoir from the packaging and prior to delivering at least the portion the second fluid medium from the fluid reservoir to the interior lumen, actuating the valve from a closed position that restricts fluid communication between the fluid reservoir and the interior lumen to an open position that permits fluid communication between the fluid reservoir and the interior lumen.

22. The method of claim 11, wherein the first fluid medium and the second fluid medium are a same type of fluid medium.

23. The method of claim 11, wherein at least one of the first fluid medium and the second fluid medium comprises an acoustic coupling fluid medium selected from the group consisting of: castor oil, refined vegetable oil, polyethylene glycol, polypropylene glycol, glycerol, saline, and Ringer's solution.

24. The method of claim 11, wherein the second fluid medium comprises microbubbles that include a therapeutic agent.

25. The method of claim 24, wherein the therapeutic agent comprises a thrombolytic drug, such as tissue plasminogen activator (tPA).

26. The method of claim 25, wherein the thrombolytic drug comprises tissue plasminogen activator (tPA).

27. The method of claim 11 wherein the second fluid medium comprises a contrast agent.

28. The method of claim 27 wherein the contrast agent is configured for use in microbubble contrast-enhanced ultrasonography.

29. The method of claim 11, wherein the fluid reservoir is a syringe that comprises a plunger movable within an interior volume of the syringe, and wherein at least the portion of the second fluid medium is delivered from the fluid reservoir to the interior lumen and at least the portion of the first fluid medium is displaced from the interior lumen through the fluid outlet by moving the plunger within the interior volume of the syringe.

30. The method of claim 11, wherein the region of interest is within a vessel, and wherein delivering the imaging catheter to the region of interest includes delivering the imaging catheter over a guidewire to the region of interest within the vessel.

31. The method of claim 11, further comprising:
receiving ultrasound energy from the region of interest at the ultrasound transducer, wherein the ultrasound energy received at the ultrasound transducer corresponds to image data of the region of interest; and
conveying the image data from the ultrasound transducer to the imaging engine.
